(19) United States
(12) Patent Application Publication
(10) Pub. No.: US 2003/0009785 A1
(43) Pub. Date: Jan. 9, 2003

(54) Plant cytoprotective genes and methods of using same

(76) Inventor: John C. Reed, Rancho Santa Fe, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/955,526

(22) Filed: Sep. 12, 2001

Related U.S. Application Data
(60) Provisional application No. 60/331,371, filed on Sep. 13, 2000.

(51) Int. Cl. A01H 5/00

(52) U.S. Cl. 800/289; 800/317.4

(57) ABSTRACT

The present invention provides a methods of increasing the resistance of a plant to biotic or abiotic stress by ectopically expressing in the plant a nucleic acid molecule encoding a plant anti-death (PAD) polypeptide or active fragment thereof. In a method of the invention, the encoded PAD polypeptide can have, for example, substantially the amino acid sequence of tomato PAD-1 (SEQ ID NO: 2) shown in FIG. 1.

This application is based on, and claims the benefit of, U.S. Provisional Application No. 60/______ (yet to be assigned), filed Sep. 13, 2000, which was converted from U.S. Ser. No. 09/661,014, and entitled PLANT CYTOPROTECTIVE GENES AND METHODS OF USING SAME, and which is incorporated herein by reference.

### BACKGROUND OF THE INVENTION

1. Field of the Invention

The present invention relates to apoptosis and plant genetic engineering and, more specifically, to the discovery of plant cytoprotective genes.

2. Background Information

Programmed cell death plays critical roles in a wide variety of normal physiological processes. Dysregulation of this natural cell death pathway contributes greatly to diseases characterized by either excessive cell accumulation such as cancer, restenosis, and autoimmunity, or inappropriate cell death such as stroke, myocardial infarction, inflammation, AIDS, Alzheimer's and other neurodegenerative diseases. In addition, most viruses and intracellular bacteria control the cell death pathway in the host cells they infect, thus linking apoptosis to infectious diseases.

In plants, as in animals, a programmed type of cell death occurs as part of normal growth and development, for example, during reproduction, seed germination, aerenchyma formation, tracheary element formation, sieve element differentiation and senescence (Pennell and Lamb, Plant Cell 9:1157-1168 (1997); Ryerson and Heath, Plant Cell 8:393-402 (1996); Jones and Dangl, Trends in Plant Science 1:114-119 (1996); and Beers, Cell Death and Differentiation 4:649-661 (1997)). Regulation of cell death pathways also occurs in plants in response to abiotic stimuli such as ultraviolet irradiation and heat (Mitsuhara et al., Curr. Biol. 9:775-778 (1997)). Moreover, cell suicide programs are activated, at least in some cases, during pathogen attack in both resistant and susceptible plants (Ryerson and Heath, Plant Cell 8:393-402 (1996) and Navarre and Wolpert, Plant Cell 11:237-249 (1999)).

The genes that control programmed cell death are conserved across wide evolutionary distances, defining a core set of biochemical reactions which are regulated in diverse ways by inputs from a variety of upstream pathways. These genes encode either anti-apoptotic or pro-apoptotic proteins, and it is the balance of these proteins that ultimately is responsible for the life-death decision of a cell. Ectopic over-expression of certain types of anti-apoptotic genes can render animal cells markedly resistant to a wide range of cell death stimuli, including nutrient deprivation, irradiation, cytotoxic chemicals, and hypoxia (Lockshin et al., Wiley-Liss, New York, 504 pp (1998)).

Elements of the same cell suicide mechanisms used in animal cells can be functionally conserved in plants. For example, expression of human anti-apoptosis genes in transgenic plants provides protection from crop-pathogens and other insults as a result of cell death suppression. Regulation of plant cell death, an area which is not well understood, is of fundamental importance for plant biology and plant genetic engineering. Exploitation of cell life/death pathways in plants can be used to improve plant crops for a variety of purposes such as to protect crops against common plant pathogens; extend post-harvest shelf-life of vegetables, fruits, and flowers; and to engineer hardier strains of plants that can survive adverse climates.

However, to date, no plant genes have been identified that share sequence homology with the anti-apoptotic genes of animal cells or which function in analogous fashion as cytoprotective gene products. Thus, there is a need to identify cytoprotective plant genes which regulate the evolutionarily conserved cell death pathway and which can be used to engineer plant varieties with improved resistant to crop pathogens and other insults. The present invention satisfies this need and provides related advantages as well.

### SUMMARY OF THE INVENTION

The present invention provides an isolated polypeptide that has an amino acid sequence encoding PAD or an active fragment thereof. An isolated polypeptide of the invention can have, for example, substantially the amino acid sequence of tomato PAD-1 (SEQ ID NO: 2), or, for example, the amino acid sequence of an ortholog of tomato PAD-1.

The invention also provides a non-naturally occurring plant that contains an ectopically expressed nucleic acid molecule encoding a PAD polypeptide or active fragment thereof and which is characterized by increased resistance to biotic or abiotic stress. In such a non-naturally occurring plant, the encoded PAD polypeptide can have, for example, substantially the amino acid sequence of tomato PAD-1 (SEQ ID NO:2), or, for example, the amino acid sequence of an ortholog of tomato PAD-1.

In one embodiment, a non-naturally occurring plant of the invention is a transgenic plant. A transgenic plant of the invention can contain, for example, an ectopically expressed nucleic acid molecule which encodes a PAD polypeptide operatively linked to an exogenous regulatory element. Such an ectopically expressed nucleic acid molecule can be an exogenous nucleic acid molecule encoding a PAD polypeptide having, for example, the amino acid sequence of an ortholog of tomato PAD-1. Exogenous regulatory elements useful in the invention include constitutive and inducible regulatory elements. Exemplary transgenic plant species include rice, corn, wheat, soybean, common fruits, turf grass and ornamental flowers. Further provided is a tissue derived from a transgenic plant of the invention. Such a tissue can be, for example, a seed or a fruit.

The present invention also provides a method of increasing the resistance of a plant to biotic or abiotic stress by ectopically expressing in the plant a nucleic acid molecule encoding a PAD polypeptide or active fragment thereof. In one embodiment, the invention provides a method of increasing the resistance of a plant to biotic or abiotic stress by introducing into the plant a nucleic acid molecule encoding a PAD polypeptide or active fragment thereof, thereby increasing the resistance of the plant to biotic or abiotic stress.

The invention also provides an isolated nucleic acid molecule that contains a nucleic acid sequence encoding a tomato Bax inhibitor-1 (BI-1) polypeptide or active fragment thereof, provided that the nucleic acid molecule is not GenBank accession AI771102. The encoded tomato BI-1 polypeptide can have, for example, substantially the amino acid sequence of tomato BI-1 (SEQ ID NO: 4) and can have the nucleic acid sequence of, for example, SEQ ID NO: 3.

The invention also provides a vector that contains a nucleic acid molecule encoding a tomato Bax inhibitor-1 (BI-1) polypeptide or active fragment thereof, provided that the nucleic acid molecule is not GenBank accession AI771102. Such a vector can be, for example, a plant expression vector. The encoded tomato BI-1 polypeptide can have, for example, substantially the amino acid sequence of tomato BI-1 (SEQ ID NO: 4).

The invention additionally provides a non-naturally occurring plant that contains an ectopically expressed nucleic acid molecule encoding a tomato Bax inhibitor-1 (BI-1) polypeptide or active fragment thereof and is characterized by increased resistance to biotic or abiotic stress. In a non-naturally occurring plant of the invention, the tomato BI-1 polypeptide can have, for example, substantially the amino acid sequence of tomato BI-1 (SEQ ID NO: 4). In a preferred embodiment, the non-naturally occurring plant is a transgenic plant. In a transgenic plant of the invention, the ectopically expressed nucleic acid molecule encoding a tomato BI-1 polypeptide can be operatively linked to an exogenous regulatory element, which can be, for example, a constitutive or inducible regulatory element. Exemplary transgenic plants encompassed by the invention include rice, corn, wheat, soybean, common fruits, turf grass and ornamental flower plants that contain an ectopically expressible nucleic acid molecule encoding a tomato BI-1 polypeptide or active fragment thereof.

The invention further provides a tissue derived from a transgenic plant that contains an ectopically expressible nucleic acid molecule encoding a tomato BI-1 polypeptide and is characterized by increased resistance to biotic or abiotic stress. Such a tissue can be, for example, a seed or a fruit.

Also provided by the invention is a method of increasing the resistance of a plant to a biotic or abiotic stress by ectopically expressing in the plant a nucleic acid molecule encoding a tomato Bax inhibitor-1 (BI-1) polypeptide or active fragment thereof. In one embodiment, the invention is practiced by introducing into the plant a nucleic acid molecule encoding a tomato BI-1 polypeptide or active fragment thereof, thereby increasing the resistance of the plant to biotic or abiotic stress.

The invention further provides an isolated polypeptide that has an amino acid sequence encoding tomato BI-1 or an active fragment thereof. Such an isolated polypeptide of the invention can have, for example, substantially the amino acid sequence of tomato BI-1 (SEQ ID NO: 4).

### BRIEF DESCRIPTION OF THE DRAWINGS

FIG. 1 shows the nucleotide (SEQ ID NO: 1) and amino acid sequence (SEQ ID NO: 2) of tomato plant anti-death-1 (PAD-1).

FIG. 2 shows the nucleotide (SEQ ID NO: 3) and amino acid sequence (SEQ ID NO: 4) of tomato Bax inhibitor-1 (BI-1).

FIG. 3 shows the evolutionarily conserved components of the cell death machinery.

### DETAILED DESCRIPTION OF THE INVENTION

The present invention relates to the discovery that plant cytoprotective genes can be used to manipulate the cell life/death pathways in plants, a discovery that fundamentally changes current strategies for optimizing crop yields, adapting crops to harsh climates, and for prolonging vegetable and produce longevity during storage and shipping. Overexpression of such a plant cytoprotective gene product can be used to produce hardier strains of plants that are protected against abiotic stresses and can survive, for example, excess heat, cold, drought, water, nutrient deprivation or excessive irradiation. Overexpression of a plant cytoprotective gene product also can be used to protect plants against common plant pathogens including viruses, fungi, bacteria, nematodes and other parasites. Thus, the discovery that two genes, plant anti-death-1 (PAD-1) and tomato Bax inhibitor-1 (BI-1), are plant cytoprotective gene products can be used to guard against crop ruin or blight while also increasing the quality and post-harvest shelf-life of vegetables, fruits, flowers and other crops, thus very positively impacting agricultural productivity.

A first cytoprotective nucleic acid molecule provided by the invention contains a nucleic acid sequence encoding a PAD polypeptide or active fragment thereof. The encoded PAD polypeptide can have, for example, substantially the amino acid sequence of SEQ ID NO: 2 and, in one embodiment, has the nucleic acid sequence of SEQ ID NO: 1. The encoded PAD polypeptide also can have, for example, the amino acid sequence of an ortholog of tomato PAD-1.

The invention also provides an isolated nucleic acid molecule that contains a nucleic acid sequence encoding a tomato BI-1 polypeptide or active fragment thereof, provided that said nucleic acid molecule is not GenBank accession AI771102. The encoded tomato BI-1 polypeptide can have, for example, substantially the amino acid sequence of tomato BI-1 (SEQ ID NO: 4) and can have the nucleic acid sequence of, for example, SEQ ID NO: 3.

Nearly all cells in multicellular organisms possess an intrinsic program for cell suicide. This program instructs the cell to eliminate itself from the organism, for the good of the overall survival of the animal or plant. Examples of cell suicide are found in unicellular bacteria and yeast (Ameisen, When Cells Die, Lockshin et al. eds., pages 3-56, Wiley-Liss, New York (1998)). During evolution of multicellular animal species, the cell suicide program was exploited for a wide variety of processes associated with fetal development including creation of body cavities, removal of redundant cells, and sculpting of body appendages and other structures. Such cell deaths that occur during development are genetically “programmed,” often requiring induction of specific genes to activate the cell death machinery, giving rise to the term “programmed cell death.” Programmed Cell Death (PCD) also has been described in plants and plays a role in specific processes in certain types of plants. These processes include seeding, deciduation of leaves, and other processes (Pennell and Lamb, Plant Cell 9:1157-1168 (1997)).

By far, the most common of the cell suicide responses in animal species is “apoptosis.” Apoptosis refers to a constellation of characteristic morphological changes that animal cells typically undergo when dying by activation of the endogenous cell suicide program. Recent research has revealed that most of these morphological and ultrastructural changes seen in apoptotic cells can be traced to the actions of a family of intracellular proteases (“caspases”) that become activated when cells undergo the cell suicide response (Villa et al., Trends Biochem. Sci. 22:388-393 (1997) and Cryns and Yuan, Genes and Development 13:1551-1568 (1999)).

In animal cells, the genes which constitute the core components of the apoptosis machinery have been identified and characterized, with the paradigm for the cell death pathway of animal cells derived largely from genetic studies in the nematode Caenorhabditis elegans. In this organism, 131 of the 1090 cells which form during development undergo programmed cell death, allowing the generation in the laboratory of genetic mutants with defects in the cell death machinery, so-called “cell death defective” (CED) mutants (Ellis et al., Annu. Rev. Cell Biol. 7:663-698 (1991)). In the worm, all developmental cell deaths are dependent on CED-3, which is a caspase-family cell death protease (Chen et al., Science 287:1485-1489 (2000)). The activation of CED-3 requires CED-4, an ATPase which directly binds the CED-3 zymogen and triggers proteolytic autoprocessing of pro-CED-3, thus generating autonomously active CED-3 protease. The actions of CED-4 are suppressed by CED-9, an anti-apoptotic protein that binds CED-4 and prevents it from activating the pro-CED-3 zymogen. In some cells of the worm, CED-9 is under negative control by EGL-1, a protein that binds CED-9 and prevents it from interacting with CED-4 (Conradt and Horvitz, Cell 93:519-529 (1998)).

The paradigm for programmed cell death elucidated in C. elegans has close parallels in humans and other animal species, and is also the target of regulation by many animal viruses (Shen and Shenk, Curr. Opin. Genet. Develop. 5:105-111 (1995)). In humans and other mammals, homologs of CED-3, CED-4, CED-9, and EGL-1 perform essentially the same functions as their counterparts in the worm. In mammalian species, however, the greater diversity of tissues and biological processes is accompanied by greater diversity in the homologs of the cell death pathway genes. Thus, the caspases, which are CED-3 homologs, exist as a multigene family of at least 14 members (Earnshaw et al., Annu. Rev. Biochem. 68:383-424 (1999)). Similarly, the Bcl-2 family contains at least five members which function analogously to CED-9 in blocking cell death (Reed, Oncogene 17:3225-3236 (1998)). At least six functional homologs of EGL-1 exist in humans; these homologs dimerize with Bcl-2 family proteins and suppress their functions. Humans also contain at least five members of a family of anti-apoptotic proteins termed the IAPs, for “Inhibitor of Apoptosis Proteins”. IAP-family proteins function as endogenous protease inhibitors, binding directly to and inhibiting certain caspases (Deveraux and Reed, Genes Develop. 13:239-252 (1999)).

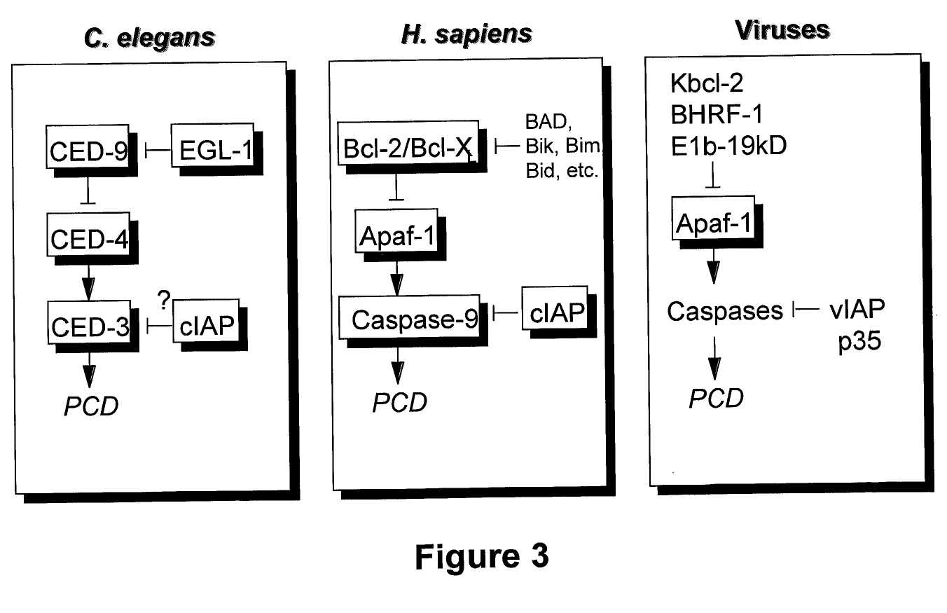

The evolutionarily conserved components of the cell death machinery depicted on the left in FIG. 3 define the core of the cell death pathway in animal cells, including humans, farm animals, and insects which prey on crops. The presence of multigene families of cell death regulators in higher organisms provides opportunities for generating tissue-specificity of apoptosis regulation. Each member of the family has its own unique pattern of expression in particular types of cells throughout the body, with certain members performing critical non-redundant functions in specific types of cells but not others.

Mitochondria are essential for apoptotic destruction of exogenous nuclei in Xenopus egg extracts, indicating the existence of a primitive pathway for cell death regulation that centers on mitochondria (Newmeyer et al., Cell 79:353-364 (1994)). Mitochondria release cytochrome c during apoptosis, with cytosolic cytochrome c then activating caspase-family cell death proteases by binding and turning-on the CED-4 homolog, Apaf-1 (Reed, Cell 91:559-562 (1997)). In yeast, ectopic expression of a pro-apoptotic human protein, Bax, conferred a lethal phenotype which was specifically suppressible by co-expression of anti-apoptotic proteins such as Bcl-2 (Matsuyama et al., Current Opinion in Microbiology 2:618-623 (1999)). When expressed in yeast, the Bax protein targeted mitochondria and induced cytochrome c release. Though yeast possess no caspases or CED-4 homologs, the damage induced by Bax to mitochondria nevertheless directly kills these unicellular organisms (Zha et al., Mol. Cell. Biol. 16:6494-6508 (1996)). Similarly, Bax kills mammalian cells through a caspase-independent mechanism involving direct damage to mitochondria such that the mitochondria become incapable of maintaining oxidative phosphorylation due to loss of cytochrome c, resulting in production of reactive oxygen species and cell death by necrosis rather than apoptosis (Green and Reed, Science 281:1309-1312 (1998) and Zha and Reed, J. Biol. Chem. 272:31482-31488 (1997)). These parallels in the cell death mechanisms invoked by Bax in animal cells and yeast provide further evidence for an ancient pathway for cell death (Manon et al., FEBS Letters 415:29-32 (1997)).

In tobacco cells, introduction and expression of Bax results in cell death which resembles the hypersensitive response (HR), a plant cell suicide program (Lacomme and Santa Cruz, Proc. Natl. Acad. Sci. USA 96:7956-7961 (1999)). In such plant cells, Bax localizes to plant mitochondria. When Bax is expressed in human cells, the earliest measurable change is a change in pH gradients across the inner membranes of mitochondria, suggesting a net efflux of H⁺ which results in acidification of the cytosol and alkalinization of the mitochondrial matrix (Green and Reed, Science 281:1309-1312 (1998)). These same changes in pH regulation are observed when Bax is ectopically expressed in yeast. Furthermore, pharmacological inhibitors of H⁺ transporters ameliorate the cytotoxic effects of Bax in both animal cells and yeast possibly due to the structural similarity of Bax to the pore-forming domains of certain bacterial toxins (Konisky, Annu. Rev. Microbiol. 36:125-144 (1982)).

Several Bcl-2 family proteins which target mitochondria bear a striking resemblance to certain pore-forming proteins of bacteria, including diphtheria toxin and the colicins (Minn et al., Nature 385:353-357 (1997) and Schlesinger et al., Proc. Natl. Acad. Sci. USA 94:11357-11362 (1997)). Like these other molecules, Bax can induce pores in mitochondrial membranes, resulting in cytochrome c efflux and other changes. Pore-forming molecules such as the bacterial colicins are secreted by bacteria and used as weapons against competing bacteria. The colicins exist initially in an inactive conformation when secreted, then bind receptors on competing bacteria, and undergo a voltage-dependent conformational change, allowing them to insert into membranes, where they form channels that depolarize membranes and kill targeted bacteria. In an analogous manner, Bax normally resides in the cytosol in a latent state, but translocates to mitochondrial membranes upon delivery of an apoptotic stimulus (or upon over-expression), and undergoes conformational changes associated with its insertion into mitochondrial membranes (Antonsson et al., Science 277:370-372 (1997) and Nouraini et al., Mol Cell Biol. 20:1604-1615 (2000)). Thus, these findings indicate that this ancient bacterial system of pore-forming proteins has been adapted for control of cell suicide in higher eukaryotes.

Programmed cell death plays a normal physiological role in a variety of processes in plants. Such processes include (a) deletion of cells with temporary functions such as the aleurone cells in seeds and the suspensor cells in embryos; (b) removal of unwanted cells, such as the root cap cells found in the tips of elongating plant roots and the stamen primoridia cells in unisexual flowers; (c) deletion of cells during sculpting of the plant body and formation of leaf lobes and perforations; (d) death of cells during plant specialization, such as the death of tracheary element (TE) cells; and (e) leaf senescence (Wang et al., Plant Mol. Biol. 32:1125-1134 (1996); Wang et al., Plant Cell 11:237-491 (1996); Hadfield and Bennett, Cell Death Different. 4:662-670 (1997); Fukuda, Cell Death Different. 4:684-688 (1997); Groover and Jones, Plant Physiol. 119:375-384 (1999); and Nam, Curr. Opin. Biotech. 8:200-207 (1997)).

Though the biochemical mechanisms responsible for cell suicide in plants are largely unknown, there are similarities to the programmed cell death that occurs in animal species. For example, programmed cell death in plants typically requires new gene expression, and thus can be suppressed by cycloheximide and similar inhibitors of protein or RNA synthesis (Havel and Durzan, Bot. Acta. 109:268-277 (1996)). Morphological characteristics of plant cells undergoing programmed cell death are similar to apoptosis in animals, though the presence of a cell wall around plant cells imposes certain differences. Akin to animal cells, programmed cell death in plants is associated with internucleosomal DNA fragmentation (DNA ladders) and the activation of proteases (see, for example, 2, 37, 43, 44). For instance, a gene encoding a DNA nuclease is induced during xylogenesis in Zinnia elegans, and genes encoding putative cysteine proteases are induced during tracheary element cell death in Zinnia and during senescence in Arabidopsis and tomato plants (Groover and Jones, Plant Physiol. 119:375-384 (1999); Nam, Curr. Opin. Biotech. 8:200-207 (1997); Solomon et al., The Plant Cell 11:431-444 (1999); Fukuda, Annu. Rev. Plant Physiol. Plant Mol. Biol. 47:299-325 (1996); and Mittler and Lam, Plant Physiol. 108:489-493 (1995)). However, in some cases, programmed cell death in plants does not exhibit these hallmark characteristics (Heath, Eur. J. Plant Pathol. 104:117-124 (1998)).

In addition to its role in developmental processes in plants, cell suicide plays an important role in interactions of plants with a variety of pathogens, including bacteria, fungi and viruses (Mittler and Lam, Trends in Microbiol. 4:10-15 (1996)). The best studied of these plant responses to pathogens is the “Hypersensitive Response” (HR). Upon exposure to certain pathogens, plant cells in the immediately affected area undergo a rapid cell suicide response that can kill cells near the site of infection, thereby limiting the spread of pathogens (Doke et al., “In Molecular Determinants of Plant Diseases,” eds. Nishimura et al., (Springer, Berlin) pp. 235-252 (1987)). A hypersensitive response is associated with expression of a variety of plant defense genes and the induction of programmed cell death. A hypersensitive response is usually preceded by rapid and transient responses including ion fluxes, alterations in protein phosphorylation patterns, pH changes, changes in membrane potential, release of reactive oxygen species (ROS; oxidative burst), and oxidative cross-linking of plant cell wall proteins (Richberg et al, Curr. Opin. Plant Biol. 1:480-488 (1998); Bolwell and Wojtaszek, Physiol. Mol. Plant Pathol. 51:347-366 (1997)). However, hypersensitive response cell death can be uncoupled from ROS production, as evidenced by number of elicitor-induced systems which generate ROS without cell death (Piedras et al., Mol. Plant Micro. Interact 11:1155-1166 (1998) and Bodwell, Curr. Opin. Plant Biol. 2:287-294 (1999)). The hypersensitive response can involve cysteine protease expression akin to animal programmed cell death (Del Pozo and Lam, Curr. Biol. 8:1129-1132 (1998)). Serine proteases are involved in some plant developmental programmed cell deaths such as death of tracheary element cell demise (Groover and Jones, Plant Physiol. 119:375-384 (1999)). Parallels with the animal cell death machinery are further indicated by reports that (a) the hypersensitive response induced by tobacco mosaic virus (TMV) in tobacco plants is associated with generation of caspase-like protease activity; and that (b) caspase-inhibitory peptides block bacteria-induced programmed cell death in Arabidopsis without significantly affecting the induction of hypersensitive-associated defense genes (Del Pozo and Lam, Curr. Biol. 8:1129-1132 (1998)). Furthermore, a number of plant resistance (R) genes which function against bacteria (for example, RPS2), fungi (for example, RPP5) and viruses (for example, N) have sequence similarity to the CED-4/APAF-1 family of proteins implicated in apoptosis regulation in animal cells (Van der Biezen and Jones, Curr. Biol. 8:R226-R227 (1998)).

Although plant cell suicide can be effective in limiting the spread of certain viruses, bacteria and fungi (in particular, those with a biotrophic lifestyle), it is counterproductive for limiting necrotizing pathogens that, in the case of certain bacteria and fungi, utilize the decaying cell corpse as a food base. For example, in plants which are sensitive to toxin producing necrotrophic fungi, including Fusarium moniliforme (fumonisin), Alternaric alternata (AAA toxin) and Cochliobolus victoriae (victorin), hallmark features of apoptosis have been observed (Navarre and Wolpert, Plant Cell 11:237-249 (1999) and Wang et al., Plant Cell 11:237-491 (1996)). Thus, plant programmed cell death can accompany both susceptible and resistant reactions, indicative of a common biochemical pathway.

Several experiments indicate that plants can be genetically engineered for pathogen-resistance without interfering with normal programmed cell death responses needed for plant development. For example, Mitsuhara et al. demonstrated that cytoprotective Bcl-2 family proteins from humans (BCl-X_{L}) and nematodes (CED-9), resulted in increased cellular resistance to UV irradiation and paraquat when expressed in tobacco (Mitsuhara et al., Curr. Biol. 9:775-778 (1997)). Bax, a death promoting member of the Bcl-2 family, induced cell death in a manner similar to the HR when expressed in tobacco, with no distinctive morphological changes (Lacomme and Santa Cruz, Proc. Natl. Acad. Sci. USA 96:7956-7961 (1999)). In addition, transgenic tobacco have been generated, which harbor various anti-apoptotic proteins including human Bcl-2, human Bcl-X_{L}, nematode CED-9 and baculovirus Op-IAP. When Sclerotinia sclerotiorum (Dickman and Mitra, Physiol. Mol. Plant Pathol. 41:255-263 (1992)), a necrotrophic fungal pathogen with an extremely broad host range of more than 400 species, was inoculated onto tobacco harboring these transgenes, plants were highly tolerant and in most cases, completely resistant. In contrast, wild type tobacco was highly susceptible. The fungus (which requires inoculation with a rich carbon source) grows vegetatively along the tobacco leaf surface of transgenic plants, but is unable to infect or colonize host tissue. Eventually, the fungus ceases growth, presumably by depleting the nutritional source and importantly, even with extended incubation, the fungus still fails to colonize and infect transgenic plant tissue. Similar results were observed with other necrotrophic fungi including Botrytis cinerea and Cercospora nicotianae.

The C. nicotiniae fungus synthesizes a light activated polyketide toxin, cercosporin (Daub, Phytopathology. 72:370-374 (1982)), and kills control tobacco plants within two weeks of inoculation. CED-9 and IAP containing transgenic tobacco were symptomatic but highly tolerant, and Bcl-2 expressing transgenic plants were extremely resistant. Cercosporin generates reactive oxygen species (ROS) such as singlet oxygen, which is required for induction of host cell death. Bcl-2 reportedly interferes with ROS-mediated cell death possibly by promoting the scavenging of free radicals (Hockenbery et al., Cell 75:241-251 (1997)). Consistent with Bcl-2 protecting plants from ROS, resistance to paraquat, a contact herbicide that kills plant cells by inducing the production of active oxygen species, was observed in transgenic Bcl-XL and CED-9-containing tobacco (Mitsuhara et al., Curr. Biol. 9:775-778 (1997)). Moreover, Bcl-2 expression reduced paraquat induced apoptosis in mouse cells (Fabisiak et al., Am. J. Physiol. 272:675-684 (1997)).

Resistance to viruses was also analyzed in the engineered plants. Many plant host-virus combinations produced necrotic lesions as a general disease symptom, irrespective of host genotype. Tobacco is a “local lesion” host for several such viruses, including tobacco necrosis virus (TNV) and tomato spotted wilt virus (TSWV). While typical virus-induced lesions were observed on wild-type plants, necrotic symptoms were greatly reduced or absent on the tobacco leaves expressing the cytoprotective Bcl-2 family members when inoculated with TSWV, and no lesions formed in the transgenic plants following inoculation with TNV. Western blots and ELISA assays demonstrated that the TNV and TSWV were confined to local lesions in the transgenic plants, and no detectable virus replication occurred.

Among the progeny of selfed tobacco plants, those with sensitivity to the selectable marker, kanamycin, and lacking transgene expression were susceptible to all the previous discussed fungal and viral pathogens. Kanamycin resistant progeny expressing a given transgene were resistant and identical in phenotype following viral challenge as observed in the parents.

To address the question of whether anti-apoptotic gene products directly affect cell death/resistance pathways in the plant, an additional set of transgenic tobacco were engineered to express a Bcl-X_{L} variant containing a G138A mutation. Glycine 138 is located in the conserved BH-1 domain of Bcl-X_{L} which is essential for its anti-apoptotic activity; the G138A mutation renders the Bcl-X_{L} protein non-functional (Yang et al., Cell 80:285-291 (1995)). The analogous mutation in Bcl-2, G145A, also leads to a loss of function. In ten independent transgenic tobacco plant lines, Bcl-X_{L} protected tobacco from S. sclerotiorum and B. cinerea infection, as did Bcl-2. However, tobacco plants expressing mutant Bcl-X_{L} (G138A) responded like untransformed control plants, although in some lines lesions developed slightly slower than in wild type tobacco inoculations. Selfed Bcl-X_{L} plants also showed segregation of resistance and transgene expression. None of the selfed progeny containing the G138A mutation showed resistance. Western blot analysis of these tobacco lines showed equivalent steady state protein levels in both Bcl-X_{L} (wild type) and Bcl-X_{L} (G138A) lines. Thus, these data show that resistance requires a functional Bcl-X_{L} protein.

In regard to the possibility of abnormal effects on the overall physiology of the transgenic tobacco, most transgenic plants grew, flowered and set seed in a similar manner to wild type tobacco. No detectable morphological or physiological alterations were observed. However, in some lines extremely resistant lines expressing high levels of Bcl-2, Bcl-X_{L} or CED-9, abnormalities were observed, including male sterility, stunted growth, flower deformation and altered leaf pigmentation. Transgenic lines expressing moderate levels of the transgene showed none of these altered growth patterns but retained pathogen resistance.

The three fungal pathogens assayed are necrotrophs; thus, these fungi require host plant cell death to grow, colonize and reproduce in the host milieu. The studies reported above indicate that these pathogens specifically interact with the plant by triggering host cell death pathways. Inhibition of this pathway by anti-apoptotic gene products prevents fungal infection, despite the fact that the fungus has its full complement of virulence factors. These results indicate that necrotrophic pathogens can co-opt plant host cell death pathways for successful colonization and disease development and that redirection of plant cell death pathways by necrotrophic pathogens can be essential for disease development to occur.

The present invention provides an isolated polypeptide that has an amino acid sequence encoding PAD or an active fragment thereof. An isolated polypeptide of the invention can have, for example, substantially the amino acid sequence of tomato PAD-1 (SEQ ID NO: 2), or, for example, the amino acid sequence of an ortholog of tomato PAD-1.

The invention further provides an isolated polypeptide that has an amino acid sequence encoding tomato BI-1 or an active fragment thereof. Such an isolated polypeptide of the invention can have, for example, substantially the amino acid sequence of tomato BI-1 (SEQ ID NO: 4).

As used herein, the term “isolated” means a polypeptide or nucleic acid molecule that is in a form that is relatively free from contaminating lipids, polypeptides, nucleic acids or other cellular material normally associated with the polypeptide or nucleic acid molecule in a cell.

The terms “PAD,” “PAD polypeptide” or “plant anti-death polypeptide,” as used herein, means a gene product that is structurally related to tomato PAD-1 (SEQ ID NO: 2) and that functions as does tomato PAD-1 as a cytoprotective gene product. A PAD polypeptide is characterized, in part, in that it contains multiple transmembrane domain, for example, six transmembrane domains and that it has cytoprotective activity.

The PAD and tomato BI-1 polypeptides of the invention are characterized, in part, by having cytoprotective activity. As used herein, the term “cytoprotective activity” refers to the ability to prevent cell death of a cell or organism, particularly a plant cell or plant organism. Cytoprotective activity can, for example, reduce the extent of apoptosis. Cytoprotectivity can be demonstrated in animal or plants cells in vitro or in vivo, and can be, for example, activity in protecting yeast cells against Bax-induced cell death as described in Example I. Cytoprotectivity can be demonstrated in plants in vivo, for example, in transgenic Arabidopsis or tobacco plants subjected to the fungus S. sclerotiorum, turnip crinkle virus, heat or UV light as disclosed in Example III.

A PAD polypeptide of the invention generally has at least 40% amino acid sequence identity to tomato PAD-1 (SEQ ID NO:2) over the full-length sequence, and can have 50%, 55%, 60%, 65%, 70%, 75%, 80% or more % sequence identity to tomato PAD-1 (SEQ ID NO:2). Percent amino acid identity can be determined using Clustal W version 1.7 (Thompson et al., Nucleic Acids Res. 22:4673-4680 (1994)).

Thus, it is clear to the skilled person that the term “PAD polypeptide” encompasses polypeptides with one or more naturally occurring or non-naturally occurring amino acid substitutions, deletions or insertions as compared to SEQ ID NO: 2, provided that the peptide has at least 40% amino acid identity with SEQ ID NO: 2 and retains cytoprotective activity. A PAD polypeptide can be, for example, a naturally occurring variant of tomato PAD-1 (SEQ ID NO: 2), a species homolog such as a rice, soybean, corn or wheat PAD ortholog, a PAD polypeptide mutated by recombinant techniques, and the like.

Modifications to SEQ ID NO: 2 that are encompassed within the invention include, for example, an addition, deletion, or substitution of one or more conservative or non-conservative amino acid residues; substitution of a compound that mimics amino acid structure or function; or addition of chemical moieties such as amino or acetyl groups. The activity of a modified PAD polypeptide or fragment thereof can be assayed, for example, by transforming Bax-expressing yeast with a PAD-encoding nucleic acid molecule and assaying for cytoprotective activity by assaying for yeast viability.

A particularly useful modification of a PAD polypeptide of the invention, or active fragment thereof, is a modification that confers, for example, increased stability. Incorporation of one or more D-amino acids is a modification useful in increasing stability of a polypeptide or polypeptide fragment. Similarly, deletion or substitution of lysine can increase stability by protecting against degradation.

The present invention also provides active fragments of a PAD polypeptide. As used herein, the term “active fragment” means a polypeptide fragment that has substantially the amino acid sequence of a portion of a PAD polypeptide and that retains cytoprotective activity. An active fragment of a PAD polypeptide can have, for example, substantially the amino acid sequence of a portion of tomato PAD-1 (SEQ ID NO: 2).

In one embodiment, a polypeptide of the invention has substantially the amino acid sequence of tomato PAD-1 (SEQ ID NO:2). As used herein, the term “substantially the amino acid sequence” when used in reference to a PAD polypeptide or an active fragment thereof, is intended to mean an identical sequence, or a similar, non-identical sequence that is considered by those skilled in the art to be a functionally equivalent amino acid sequence. For example, an amino acid sequence that has substantially the amino acid sequence of tomato PAD-1 (SEQ ID NO: 2) can have one or more modifications such as amino acid additions, deletions or substitutions relative to the amino acid sequence of SEQ ID NO:2, provided that the modified polypeptide retains substantially the ability to be cytoprotective.

As used herein, the term “ortholog” refers to a naturally occurring plant gene product that, of all the genes in the genome of interest, is most highly homologous to tomato PAD-1 (SEQ ID NO: 2) at the amino acid level. For example, a PAD ortholog can be, for example, rice PAD, corn PAD, or soybean PAD. A PAD ortholog retains the cytoprotective activity of the tomato PAD-1 polypeptide. An ortholog of tomato PAD-1 can be readily isolated using fragments of SEQ ID NO: 1 as primers or probes to amplify or screen a cDNA library prepared from the plant species of interest using methods well known in the art (see, for example, (Glick and Thompson (eds.), Methods in Plant Molecular Biology and Biotechnology, Boca Raton, Fla.: CRC Press (1993).

The term “tomato BI-1” or “tomato BI-1 polypeptide,” as used herein, means a plant gene product that is structurally related to tomato BI-1 (SEQ ID NO: 4) and that functions as does tomato BI-1 as a cytoprotective gene product. A tomato BI-1 polypeptide is characterized, in part, in that it contains multiple transmembrane domains, for example, six transmembrane domains and that it has cytoprotective activity. Cytoprotectivity can be demonstrated in animal or plants cells in vitro or in vivo, and can be activity in protecting yeast cells against Bax-induced cell death as described in Example I.

A tomato BI-1 polypeptide of the invention generally has at least 70% amino acid sequence identity to tomato BI-1 (SEQ ID NO:4), and can have 75%, 80%, 85%, 90%, 95% or more % sequence identity to tomato BI-1 (SEQ ID NO:4). Percent amino acid identity can be determined using Clustal W version 1.7 (Thompson et al., Nucleic Acids Res. 22:4673-4680 (1994)).

Thus, it is clear to the skilled person that the term “tomato BI-1” encompasses polypeptides with one or more naturally occurring or non-naturally occurring amino acid substitutions, deletions or insertions as compared to SEQ ID NO: 4, provided that the peptide has at least 70% amino acid identity with SEQ ID NO: 5 and retains cytoprotective activity. A tomato BI-1 polypeptide can be, for example, a naturally occurring variant of tomato BI-1 (SEQ ID NO: 4), an alternatively spliced form, a tomato BI-1 polypeptide mutated by recombinant techniques, and the like.

Modifications to SEQ ID NO: 4 that are encompassed within the invention include, for example, an addition, deletion, or substitution of one or more conservative or non-conservative amino acid residues; substitution of a compound that mimics amino acid structure or function; or addition of chemical moieties such as amino or acetyl groups. The activity of a modified tomato BI-1 polypeptide or fragment thereof can be assayed, for example, by transforming Bax-expressing yeast with a tomato BI-1-encoding nucleic acid molecule and assaying for cytoprotective activity by assaying for yeast viability.

A particularly useful modification of a tomato BI-1 polypeptide of the invention, or active fragment thereof, is a modification that confers, for example, increased stability. Incorporation of one or more D-amino acids is a modification useful in increasing stability of a polypeptide or polypeptide fragment. Similarly, deletion or substitution of lysine can increase stability by protecting against degradation.

The present invention also provides active fragments of a tomato BI-1 polypeptide. As used herein, the term “active fragment” means a polypeptide fragment that has substantially the amino acid sequence of a portion of a tomato BI-1 polypeptide and that retains cytoprotective activity. An active fragment of a tomato BI-1 polypeptide can have, for example, substantially the amino acid sequence of a portion of tomato BI-1 (SEQ ID NO: 4).

In one embodiment, a polypeptide of the invention has substantially the amino acid sequence of tomato BI-1 (SEQ ID NO: 4). As used herein, the term “substantially the amino acid sequence” when used in reference to a tomato BI-1 polypeptide or an active fragment thereof, is intended to mean an identical sequence, or a similar, non-identical sequence that is considered by those skilled in the art to be a functionally equivalent amino acid sequence. For example, an amino acid sequence that has substantially the amino acid sequence of tomato BI-1 (SEQ ID NO: 4) can have one or more modifications such as amino acid additions, deletions or substitutions relative to the amino acid sequence of SEQ ID NO:4, provided that the modified polypeptide retains substantially the ability to be cytoprotective.

An active fragment of a PAD polypeptide or tomato BI-1 polypeptide also is encompassed by the invention. Such an active fragment has the cytoprotective activity of the parent polypeptide as assayed in vitro or in vivo. Thus, an active fragment of a PAD polypeptide or tomato BI-1 can have activity, for example, in protecting yeast cells against cell death induced by Bax expression in transformed yeast, as described in Example I. A fragment of a PAD polypeptide or tomato BI-1 also can be shown to have cytoprotective activity by protection of a transgenic plant ectopically expressing the fragment from a biotic stress such as a virus, fungus, bacterium, nematode or other parasites, or from an abiotic stress such as heat, cold, drought, flood, nutrient deprivation or irradiation such as UV light.

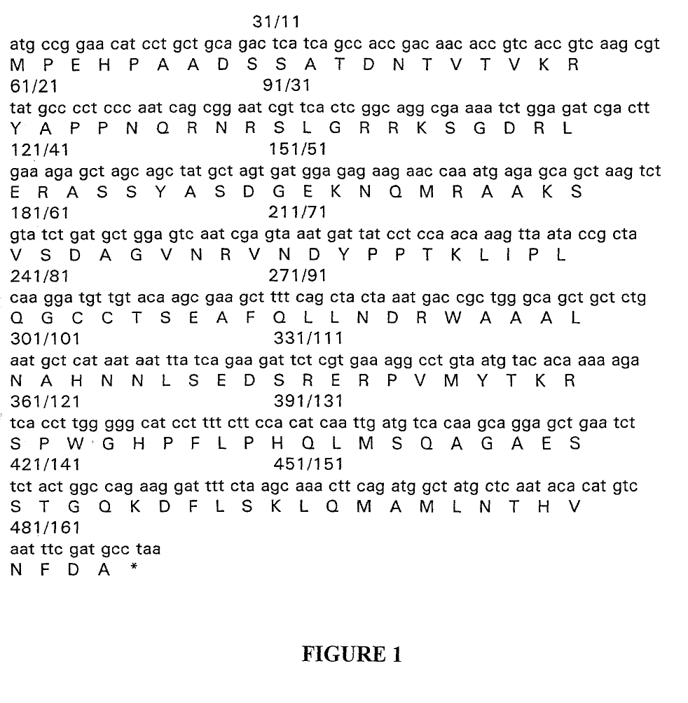

The present invention provides an isolated nucleic acid molecule that contains a nucleic acid sequence encoding a PAD polypeptide or active fragment thereof. A nucleic acid molecule of the invention can encode, for example, a PAD polypeptide having substantially the amino acid sequence of tomato PAD-1 (SEQ ID NO: 2) or a PAD polypeptide having the amino acid sequence of an ortholog of tomato PAD-1. In one embodiment, a nucleic acid molecule of the invention encodes the amino acid sequence of tomato PAD-1 (SEQ ID NO: 2) and can have, for example, the nucleic acid sequence of SEQ ID NO: 1 (see FIG. 1).

The invention also provides an isolated nucleic acid molecule that contains a nucleic acid sequence encoding a tomato BI-1 polypeptide or active fragment thereof, provided that the nucleic acid molecule is not GenBank accession AI771102. The encoded tomato BI-1 polypeptide can have, for example, substantially the amino acid sequence of tomato BI-1 (SEQ ID NO: 4) and can have the nucleic acid sequence of, for example, SEQ ID NO: 3.

As used herein, the term “nucleic acid molecule” means any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded. Such molecules can be, for example, DNA or RNA, or a DNA/RNA hybrid.

Further provided by the invention is an oligonucleotide that contains a nucleotide sequence having at least 8 contiguous nucleotides of SEQ ID NO: 1, or a nucleotide sequence complementary thereto. Such an oligonucleotide can have, for example, at least 10 or 15 contiguous nucleotides of SEQ ID NO: 1, or a nucleotide sequence complementary thereto.

Also provided by the invention is an oligonucleotide that contains a nucleotide sequence having at least 8 contiguous nucleotides of SEQ ID NO: 3, or a nucleotide sequence complementary thereto, provided that the oligonucleotide sequence does not consist of a sequence of GenBank accession number AI771102. Such an oligonucleotide can have, for example, at least 10 or 15 contiguous nucleotides of SEQ ID NO: 3, or a nucleotide sequence complementary thereto.

Oligonucleotides of the invention can advantageously be used, for example, as primers for PCR or sequencing, as probes for diagnostic and other assays, and in therapeutic methods. An oligonucleotide of the invention can incorporate, if desired, a detectable moiety such as a radiolabel, fluorochrome, luminescent tag, ferromagnetic substance, or a detectable agent such as biotin, and can be useful, for example, for detecting mRNA expression of a PAD polypeptide or a BI-1 polypeptide in a cell or tissue and for Southern analysis. Those skilled in the art can determine the appropriate length of a PAD or BI-1 oligonucleotide for a particular application. An oligonucleotide of the invention contains a nucleotide sequence having, for example, at least 8, 10, 12, 14, 16, 18, 20, 25, 30, 35, 40, 50, 100 or 200 contiguous nucleotides of SEQ ID NO: 1 or of SEQ ID NO: 3, or a nucleotide sequence complementary thereto.

The invention also provides an isolated antisense nucleic acid molecule which contains a nucleotide sequence that specifically binds to SEQ ID NO: 1 or 3, provided that the nucleic acid molecule is not GenBank accession AI771102 or the complement thereof. Such an isolated antisense nucleic acid molecule can have, for example, at least 20 nucleotides complementary to SEQ ID NO: 1 or 3 and can have, for example, at least 25, 30, 35, 40, 45, 50 or more nucleotides complementary to SEQ ID NO: 1 or SEQ ID NO: 3. In one embodiment, an isolated antisense nucleic acid molecule has at least 20 nucleotides complementary to SEQ ID NO: 1 or SEQ ID NO: 3 and contains a nucleotide sequence complementary to the sequence “ATG,” provided that the nucleic acid molecule is not GenBank accession AI771102 or the complement thereof.

An antisense nucleic acid molecule of the invention specifically binds to the nucleotide sequence of SEQ ID NO:1 or 3. An antisense nucleic acid molecule that “specifically binds” SEQ ID NO: 1 or SEQ ID NO: 3 binds with substantially higher affinity to that particular nucleotide sequence than to an unrelated nucleotide sequence.

A nucleic acid molecule of the invention, including a sense or antisense nucleic acid molecule, or an oligonucleotide of the invention, also can contain one or more nucleic acid analogs. Nucleoside analogs or phosphothioate bonds protect against degradation by nucleases are particularly useful in a nucleic acid molecule or oligonucleotide of the invention. A ribonucleotide containing a 2-methyl group, instead of the normal hydroxyl group, bonded to the 2′-carbon atom of ribose residues, is an example of a non-naturally occurring RNA molecule that is resistant to enzymatic and chemical degradation. Other examples of non-naturally occurring organic molecules include RNA containing 2′-aminopyrimidines, such RNA being 1000× more stable in human serum as compared to naturally occurring RNA (see Lin et al., Nucl. Acids Res. 22:5229-5234 (1994); and Jellinek et al., Biochemistry 34:11363-11372 (1995)).

Additional nucleotide analogs also are well known in the art. For example, RNA molecules containing 2′-O-methylpurine substitutions on the ribose residues and short phosphorothioate caps at the 3′- and 5′-ends exhibit enhanced resistance to nucleases (Green et al., Chem. Biol. 2:683-695 (1995)). Similarly, RNA containing 2′-amino- 2′-deoxypyrimidines or 2′-fluro- 2′-deoxypyrimidines is less susceptible to nuclease activity (Pagratis et al., Nature Biotechnol. 15:68-73 (1997)). Furthermore, L-RNA, which is a stereoisomer of naturally occurring D-RNA, is resistant to nuclease activity (Nolte et al., Nature Biotechnol. 14:1116-1119 (1996)); Klobmann et al., Nature Biotechnol. 14:1112-1115 (1996)). Such RNA molecules and methods of producing them are well known and routine in the art (see Eaton and Piekern, Ann. Rev. Biochem. 64:837-863 (1995)). DNA molecules containing phosphorothioate linked oligodeoxynucleotides are nuclease resistant (Reed et al., Cancer Res. 50:6565-6570 (1990)). Phosphorothioate-3′ hydroxypropylamine modification of the phosphodiester bond also reduces the susceptibility of a DNA molecule to nuclease degradation (see Tam et al., Nucl. Acids Res. 22:977-986 (1994)). Furthermore, thymidine can be replaced with 5-(1-pentynyl)-2′-deoxoridine (Latham et al., Nucl. Acids Res. 22:2817-2822 (1994)). It is understood that nucleic acid molecules, including antisense molecules and oligonucleotides, containing one or more nucleotide analogs or modified linkages are encompassed by the invention.

Also provided by the invention is a vector that contains a nucleic acid molecule encoding a PAD polypeptide or active fragment thereof. Such a vector can be, for example, a plant expression vector and can encode a PAD polypeptide having, for example, substantially the amino acid sequence of tomato PAD-1 (SEQ ID NO: 2), or the amino acid sequence of an ortholog of tomato PAD-1. In addition to a nucleic acid molecule encoding the PAD polypeptide, a vector also can contain, for example, one or more regulatory elements that control expression of the PAD polypeptide-encoding nucleic acid molecule.

The invention also provides a vector that contains a nucleic acid molecule encoding a tomato BI-1 polypeptide or active fragment thereof, provided that the nucleic acid molecule is not GenBank accession AI771102. Such a vector can be, for example, a plant expression vector. The encoded tomato BI-1 polypeptide can have, for example, substantially the amino acid sequence of tomato BI-1 (SEQ ID NO: 4).

A variety of regulatory elements are useful in the vectors and transgenic plants of the invention including constitutive, inducible and tissue-selective or tissue-specific regulatory elements. A constitutive regulatory element can be useful in a vector of the invention, or in a transgenic plant of the invention as described further below. As used herein, the term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant regulatory element typically is expressed independently of the developmental stage of the plant, and independently of the conditions under which the plant is grown. A constitutive regulatory element that is expressed in a plant generally is widely expressed in a large number of cell and tissue types in the plant.

A variety of constitutive regulatory elements useful in a plant expression vector or transgenic plant of the invention are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous and diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other constitutive regulatory elements useful in a transgenic plant of the invention include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient ectopic expression in monocots also are known in the art including, for example, the pEmu promoter and promoters based on the rice actin-1 5′ region (Last et al., Theor. Appl. Genet. 81:581 (1991); McElroy et al., Mol. Gen. Genet. 231:150 (1991); McElroy et al., Plant Cell 2:163 (1990)). Chimeric regulatory elements, which combine elements from different genes, also can be useful for ectopically expressing a nucleic acid molecule encoding a plant cytoprotective polypeptide such as the tomato PAD-1 polypeptide or tomato BI-1 (Comai et al., Plant Mol. Biol. 15:373 (1990)). One skilled in the art understands that a particular constitutive regulatory element is chosen based, in part, on the plant species in which an exogenous nucleic acid molecule is to be ectopically expressed and on the desired level of expression.

An exogenous regulatory element useful in a vector or transgenic plant of the invention also can be an inducible regulatory element, which is a regulatory element that confers conditional expression upon an operatively linked nucleic acid molecule, where expression of the operatively linked nucleic acid molecule is increased in the presence of a particular inducing agent or stimulus as compared to expression of the nucleic acid molecule in the absence of the inducing agent or stimulus. Particularly useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)).

An inducible regulatory element useful in a transgenic plant of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)). Additional inducible regulatory elements include salicylic acid inducible regulatory elements (Uknes et al., Plant Cell 5:159-169 (1993); Bi et al., Plant J. 8:235-245 (1995)); plant hormone-inducible regulatory elements (Yamaguchi-Shinozaki et al., Plant Mol. Biol. 15:905 (1990); Kares et al., Plant Mol. Biol. 15:225 (1990)); and human hormone-inducible regulatory elements such as the human glucocorticoid response element (Schena et al., Proc. Natl. Acad. Sci. USA 88:10421 (1991)).

In a preferred embodiment, the invention provides a plant expression vector containing a nucleic acid molecule encoding a plant cytoprotective polypeptide such as tomato PAD-1 or tomato BI-1, or an active fragment of a plant cytoprotective polypeptide under control of a leaf/stalk tissue-selective regulatory element. Since such a regulatory element is not expressed in the fruit, which in many plants is the part ingested by humans. Exemplary regulatory elements that are selective for the leaf or stalks of plants include leaf-specific promoters such as the rubisco and chlorophyll a/b binding proteins promoters; root specific regulatory elements derived from root-specific genes (Depater and Schhilperoort, Plant. Mol. Biol. 18:161 (1992); Vanderzaal et al., Plant Mol. Biol. 16:983 (1991); and Oppenheimer et al., Gene 63:87 (1988)) also can be useful in the methods of the invention.

The present invention further provides a non-naturally occurring plant that contains an ectopically expressed nucleic acid molecule encoding a PAD polypeptide or active fragment thereof, where the non-naturally occurring plant is characterized by increased resistance to biotic or abiotic stress. In such a non-naturally occurring plant, the encoded PAD polypeptide can have, for example, substantially the amino acid sequence of tomato PAD-1 (SEQ ID NO:2), or, for example, the amino acid sequence of an ortholog of tomato PAD-1.

In one embodiment, a non-naturally occurring plant of the invention is a transgenic plant that contains, for example, an ectopically expressed nucleic acid molecule which encodes a PAD polypeptide operatively linked to an exogenous regulatory element and is characterized by increased resistance to biotic or abiotic stress. Such an ectopically expressed nucleic acid molecule can be an exogenous nucleic acid molecule encoding a PAD polypeptide having the amino acid sequence of an ortholog of tomato PAD-1. Exogenous regulatory elements useful in the invention include constitutive and inducible regulatory elements. Exemplary transgenic plant species include rice, corn, wheat, soybean, common fruits, turf grass and ornamental flowers.

The invention additionally provides a non-naturally occurring plant that contains an ectopically expressed nucleic acid molecule encoding a tomato BI-1 polypeptide or active fragment thereof and is characterized by increased resistance to biotic or abiotic stress. In a non-naturally occurring plant of the invention, the tomato BI-1 polypeptide can have, for example, substantially the amino acid sequence of tomato BI-1 (SEQ ID NO: 4). In a preferred embodiment, the non-naturally occurring plant is a transgenic plant. In a transgenic plant of the invention, the ectopically expressed nucleic acid molecule encoding a tomato BI-1 polypeptide can be operatively linked to an exogenous regulatory element, which can be, for example, a constitutive or inducible regulatory element. Exemplary transgenic plants that ectopically express a tomato BI-1 polypeptide and are encompassed by the invention include rice, corn, wheat, soybean, common fruits, turf grass and ornamental flower plants.

A non-naturally occurring plant of the invention is characterized by increased resistance to biotic or abiotic stress. Thus, in comparison with a corresponding plant that is genetically similar but which does not ectopically express a nucleic acid molecule encoding a PAD polypeptide, or tomato BI-1 polypeptide, a non-naturally occurring plant demonstrates fewer symptoms in response to one or more biotic or abiotic stresses. Typical biotic stresses are common plant pathogens such as viruses, fungi, bacteria, nematodes and other parasites. Typical abiotic stresses are excess heat, cold, drought, water, nutrient deprivation or excessive irradiation. It is understood that a non-naturally occurring plant of the invention can exhibit increased resistance to one or more but not necessarily all biotic and abiotic stresses. It further is understood that an significant decrease in the severity of symptoms in response to the biotic or abiotic stress is indicative of “increased resistance.”

A non-naturally occurring transgenic plant of the invention, such as a transgenic plant, can be one of a variety of diverse plant species and can be an angiosperm or gymnosperm. An angiosperm is a seed-bearing plant whose seeds are borne in a mature ovary (fruit) and is recognized commonly as a flowering plant. Angiosperms are divided into two broad classes based on the number of cotyledons, which are seed leaves that generally store or absorb food. Thus, a monocotyledonous angiosperm is an angiosperm having a single cotyledon, whereas a dicotyledonous angiosperm is an angiosperm having two cotyledons. A variety of angiosperms are known including, for example, oilseed plants, leguminous plants, fruit-bearing plants, ornamental flowers, cereal plants and hardwood trees, which general classes are not necessarily exclusive. The skilled artisan will recognize that a cytoprotective gene of the invention can be expressed in one of these or another angiosperm, as desired. A cytoprotective gene of the invention also can be expressed in a gymnosperm, which is a seed-bearing plant with seeds not enclosed in an ovary.

The Fabaceae encompass both grain legumes and forage legumes. Grain legumes include, for example, soybean (glycine), pea, chickpea, moth bean, broad bean, kidney bean, lima bean, lentil, cowpea, dry bean and peanut. Forage legumes include alfalfa, lucerne, birdsfoot trefoil, clover, stylosanthes species, lotononis bainessii and sainfoin. The skilled artisan will recognize that any member of the Fabaceae can be modified as disclosed herein to produce a non-naturally occurring plant of the invention characterized by delayed seed dispersal.

A non-naturally occurring plant of the invention also can be, for example, one of the monocotyledonous grasses, which produce many of the valuable small-grain cereal crops of the world. Such a grass plant can be, for example, a small grain cereal plant, such as a barley, wheat, oat, rye, guinea grass, sorghum or turf grass plant. A variety of turf grass plants are known to those skilled in the art and include, for example, Kentucky bluegrass, perennial ryegrass, tall fescue, fine fescue, zoysiagrass, bahiagrass, St. Augustinegrass and buffalograss.

A variety of common fruits are known in the art. As known in the art, “common fruits” means a fruit suitable for human consumption. The term common fruits includes but is not limited to, apples; oranges, grapefruit, lemons, limes and other citrus fruits; pears, peaches, plums; blueberries, raspberries, strawberries and other berries; cantaloupe, watermelon and other melon; grapes, papaya, mango and banana.

The invention also provides a transgenic plant in which a nucleic acid molecule encoding a plant cytoprotective polypeptide is ectopically expressed and which is characterized by increased resistance to biotic or abiotic stress. In a transgenic plant of the invention, the ectopically expressed nucleic acid molecule encoding the plant cytoprotective polypeptide can be operatively linked to an exogenous regulatory element.

As used herein, the term “non-naturally occurring,” when used in reference to a plant, means a plant that has been genetically modified by man. A transgenic plant of the invention, for example, is a non-naturally occurring plant that contains an exogenous nucleic acid molecule and, therefore, has been genetically modified by man. In addition, a plant that contains, for example, a mutation in an endogenous PAD regulatory element or coding sequence as a result of calculated exposure to a mutagenic agent, such as a chemical mutagen, or an “insertional mutagen,” such as a transposon, also is considered a non-naturally occurring plant, since it has been genetically modified by man. In contrast, a plant containing only spontaneous or naturally occurring mutations is not a “non-naturally occurring plant” as defined herein and, therefore, is not encompassed within the invention. One skilled in the art understands that, while a non-naturally occurring plant typically has a nucleotide sequence that is altered as compared to a naturally occurring plant, a non-naturally occurring plant also can be genetically modified by man without altering its nucleotide sequence, for example, by modifying its methylation pattern.

The term “ectopically,” as used herein in reference to expression of a nucleic acid molecule encoding a polypeptide, refers to an expression pattern that is distinct from the expression pattern in a wild type plant. Thus, one skilled in the art understands that ectopic expression of a nucleic acid encoding, for example, a PAD polypeptide, can refer to expression in a cell type other than a cell type in which the nucleic acid molecule normally is expressed, or at a time other than a time at which the nucleic acid molecule normally is expressed, or at a level other than the level at which the nucleic acid molecule normally is expressed. Thus, overexpression, whether constitutive or inducible, is an example of “ectopic expression.”

As used herein, the term “transgenic” refers to a plant that contains an exogenous nucleic acid molecule, which can be derived from the same plant species or a heterologous plant species.

The term “exogenous,” as used herein in reference to a nucleic acid molecule and a transgenic plant, means a nucleic acid molecule originating from outside the plant. An exogenous nucleic acid molecule can have a naturally occurring or non-naturally occurring nucleotide sequence and can be a heterologous nucleic acid molecule derived from a different plant species than the plant into which the nucleic acid molecule is introduced, or can be a nucleic acid molecule derived from the same plant species as the plant into which it is introduced.

The term “operatively linked,” as used in reference to a regulatory element and a nucleic acid molecule, means that the regulatory element confers regulated expression upon the operatively linked nucleic acid molecule. It is recognized that a regulatory element and a nucleic acid molecule that are operatively linked have, at a minimum, all elements essential for transcription, including, for example, a TATA box.

It should be recognized that a non-naturally occurring plant of the invention, which contains an ectopically expressed nucleic acid molecule encoding one of the plant cytoprotective polypeptides disclosed herein, also can contain one or more additional modifications, including naturally and non-naturally occurring modifications or transgenes, that can modulate or accentuate the effect of the cytoprotective polypeptide. For example, a transgene encoding a cytoprotective PAD or tomato BI-1 polypeptide of the invention can be combined in a plant with a second transgene with anti-apoptotic activity, for example, human Bcl-2, human Bcl-X_{L}, nematode CED-9 or baculovirus Op-IAP. For example, tomato or other BI-1 can be ectopically expressed together with PAD-1, or Bcl-2 such as human Bcl-2 can be ectopically expressed together with tomato BI-1 to produce a transgenic plant in which the resulting increased resistance to biotic or abiotic stress is additive or synergistic.

The invention further provides a tissue derived from a transgenic plant of the invention that contains an ectopically expressible nucleic acid molecule encoding a PAD polypeptide and that is characterized by increased resistance to biotic or abiotic stress. Such a tissue can be, for example, a seed or a fruit.

The invention further provides a tissue derived from a transgenic plant that contains an ectopically expressible nucleic acid molecule encoding a tomato BI-1 polypeptide and that is characterized by increased resistance to biotic or abiotic stress. Such a tissue can be, for example, a seed or a fruit.

As used herein, the term “tissue” means an aggregate of plant cells and intercellular material organized into a structural and functional unit. A particular useful tissue of the invention is a tissue that can be vegetatively or non-vegetatively propagated such that the plant from which the tissue was derived is reproduced. A tissue of the invention can be, for example, a seed, leaf, root or part thereof.

As used herein, the term “seed” means a structure formed by the maturation of the ovule of a plant following fertilization. Such seeds can be readily harvested from a non-naturally occurring plant of the invention.

The present invention also provides a method of increasing the resistance of a plant to biotic or abiotic stress by ectopically expressing in the plant a nucleic acid molecule encoding a PAD polypeptide or active fragment thereof. In one embodiment, the invention provides a method of increasing the resistance of a plant to biotic or abiotic stress by introducing into the plant a nucleic acid molecule encoding a PAD polypeptide or active fragment thereof, thereby increasing the resistance of the plant to biotic or abiotic stress.

Also provided by the invention is a method of increasing the resistance of a plant to biotic or abiotic stress by ectopically expressing in the plant a nucleic acid molecule encoding a tomato BI-1 polypeptide or active fragment thereof. In one embodiment, the invention is practiced by introducing into the plant a nucleic acid molecule encoding a tomato BI-1 polypeptide or active fragment thereof, thereby increasing resistance of the plant to biotic or abiotic stress.

An exogenous nucleic acid molecule encoding, for example, tomato PAD-1 (SEQ ID NO: 2) or tomato BI-1 (SEQ ID NO: 4) can be introduced into a plant for ectopic expression using a variety of transformation methodologies including Agrobacterium-mediated transformation and direct gene transfer methods such as electroporation and microprojectile-mediated transformation (see, generally, Wang et al. (eds), Transformation of Plants and Soil Microorganisms, Cambridge, UK: University Press (1995), which is incorporated herein by reference). Transformation methods based upon the soil bacterium Agrobacterium tumefaciens are particularly useful for introducing an exogenous nucleic acid molecule into a plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or, preferably, binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art (Glick and Thompson, supra, 1993). Wounded cells within the plant tissue that have been infected by Agrobacterium can develop organs de novo when cultured under the appropriate conditions; the resulting transgenic shoots eventually give rise to transgenic plants that ectopically express a nucleic acid molecule encoding an AGL8-like gene product. Agrobacterium also can be used for transformation of whole seed plants as described in Bechtold et al., C.R. Acad. Sci. Paris, Life Sci. 316:1194-1199 (1993), which is incorporated herein by reference). Agrobacterium-mediated transformation is useful for producing a variety of transgenic seed plants (Wang et al., supra, 1995) including, for example, soybean, pea, lentil and bean.

Microprojectile-mediated transformation also can be used to produce a transgenic seed plant that ectopically expresses a PAD polypeptide or a tomato BI-1 polypeptide, or an active fragment of one of these cytoprotective polypeptides. This method, first described by Klein et al. (Nature 327:70-73 (1987), which is incorporated herein by reference), relies on microprojectiles such as gold or tungsten that are coated with the desired nucleic acid molecule by precipitation with calcium chloride, spermidine or PEG. The microprojectile particles are accelerated at high speed into an angiosperm tissue using a device such as the BIOLISTIC PD-1000 (Biorad; Hercules Calif.).

Microprojectile-mediated delivery or “particle bombardment” is especially useful to transform seed plants that are difficult to transform or regenerate using other methods. Microprojectile-mediated transformation has been used, for example, to generate a variety of transgenic plant species, including cotton, tobacco, corn, hybrid poplar and papaya (see Glick and Thompson, supra, 1993) as well as cereal crops such as wheat, oat, barley, sorghum and rice (Duan et al., Nature Biotech. 14:494-498 (1996); Shimamoto, Curr. Opin. Biotech. 5:158-162 (1994), each of which is incorporated herein by reference). In view of the above, the skilled artisan will recognize that Agrobacterium-mediated or microprojectile-mediated transformation, as disclosed herein, or other methods known in the art can be used to introduce a nucleic acid molecule encoding a PAD polypeptide, or active fragment thereof, or tomato BI-1 polypeptide, or active fragment thereof, into a plant for ectopic expression.

The following examples are intended to illustrate but not limit the present invention.

### EXAMPLE I

### Functional Screening for Plant BAX-Inhibitors

This example describes the cloning of plant Bax inhibitors using a functional screening assay in yeast.

The major function-based screen described below is predicated on the ability of ectopically expressed mammalian Bax to kill yeast and on the ability of cytoprotective proteins to rescue yeast from the lethal phenotype conferred by Bax. This screening approach has been used previously to identify Bax-inhibiting genes (Xu and Reed, “Bax inhibitor-1, a mammalian apoptosis suppressor identified by functional screening in yeast,” Mol. Cell 1:337-46 (1998); Xu et al., “Exploiting yeast for the investigation on mammalian proteins that regulate programmed cell death.” In: Zhu and Chun (eds.), Apoptosis detection and assay methods, pp. 93-115. Natick: BioTechniques Books (1998); Xu et al., Methods 17:292-304 (1999); Zhang et al., Proc. Natl. Acad. Sci. USA 97:2597-2602 (2000)).

As an overview, GAL10-bax-containing yeast cells were grown in glucose-containing medium (to repress Bax expression), and these cells were transformed with libraries of plant cDNAs under the control of the S. cerevisiae ADH1 promoter that mediates high-level gene expression in yeast. Transformants are selected by plating transformed cells on galactose-containing solid medium to induce Bax expression. Those viable transformants that grew were presumed to contain cDNAs, which by overexpression can suppress the Bax cytotoxicity. Since a yeast transformant may contain several different types of plasmids, the cDNA that is actually responsible for suppressing Bax toxicity is segregated from other irrelevant cDNAs by “passing-through-E. coli.” The ability of the cDNA to neutralize Bax cytotoxicity was verified by re-introducing the cDNA into GAL10-bax-bearing yeast cells. Since some of the cDNAs can encode proteins that somehow interfere with the expression of the Gal10 promoter as opposed to blocking the function of Bax, each candidate clone was tested for suppression of a Gal10-lacZ gene. Those cDNAs that test positive for suppression of Bax function but not Gal10 promoter expression, were then taken forward to immunoblot analysis, to verify that they do not interfere with Bax protein production in yeast.

Two tomato cDNA libraries (in pJG4-5) were transformed into Bax-containing yeast strain QX95001 and plated onto SC-L-T/galactose plates. After six days of incubation at 30° C., hundreds of colonies grew up in the galactose selective plates from 5×10⁶ transformants.

Of the transformants, 114 colonies were picked and re-streaked onto SC-L-T/galactose plates and incubated at 30° C. for three days. Of these, 61 showed growth on the galactose media. Total DNA was prepared from all 61 yeast strains by standard procedures. The library plasmids were rescued into bacteria by transformation, and plasmid DNA was prepared.

The 61 library plasmids were retransformed back into Bax-containing yeast strain QX95001 and plated onto SC-L-T/glucose plates. After two days' incubation at 30° C., three colonies from each transformation were picked and streaked onto SC-L-T/galactose plates. After three days' incubation, colonies from only eight of the 61 transformation grew on galactose plates.

These eight library plasmids were then transformed into yeast strain EGY48 with pGilda-Bax. Transformants were grew in SC-T-H/galactose media, and total protein extracts made and analyzed by Western blotting with Lex-A antibody. All showed similar expression level compared to empty vector control, indicating that Bax-expression was not affected by the library plasmids.

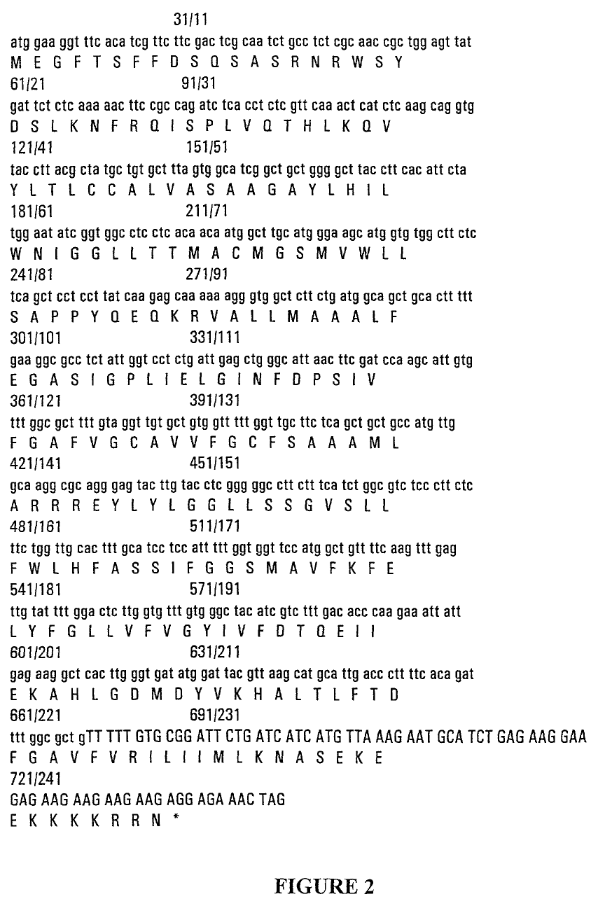

Sequencing of the eight plasmids was performed by standard methods. Two of the plasmids were the tomato PAD-1 gene shown in FIG. 1 and the BI-1 homolog shown in FIG. 2.

### EXAMPLE II

### Preparation of Mammalian Expression Constructs

This example describes the preparation of expression vectors for expression of tomato PAD-1 (SEQ ID NO: 2) shown in FIG. 1 and tomato BI-1 (SEQ ID NO: 4) shown in FIG. 2.

For expression in mammalian cells, vector pcDNA3-myc vector (Stratagene) was used for expression of the plant genes with N-terminal myc tags under control of the CMV promoter. These constructs can be monitored using commercially available myc-antibody. Primers NKO128 (GGAATTCATGGAAGGTTTCACATCGTTC; SEQ ID NO: 5) and NKO129 (CCGCTCGAGCTAGGGTCGACTGTTTCTCCTCTTC TTCTTCTTC; SEQ ID NO: 6) were used to amplify the full-length tomato BI-1 sequence. Primers NKO123 (GGAATTCATGCCGGAACATCCTGCTGCA; SEQ ID NO: 7) and T7 were used for the cloning of full-length PAD-1. PCR products were digested with EcoRI and XhoI and then cloned into the corresponding sites of pcDNA3-myc.

### EXAMPLE III

### Assays for Increased Resistance to Pathogens and Abiotic Insults

Transgenic Arabidopsis or tobacco plants ectopically expressing tomato PAD-1 (SEQ ID NO: 2) or tomato BI-1 (SEQ ID NO: 4) are evaluated for enhanced resistance to pathogens and abiotic insults as follows:

A. Pathogen Resistance

All Arabidopsis transgenic plants are evaluated initially for fungal and viral resistance. S. sclerotiorum is the primary fungus used, and the extensively characterized turnip crinkle virus is used to assay for resistance to viral infections. Additional pathogens assayed include obligate parasites such as Peronospora parasitica.

B. Abiotic Insults

Abiotic stresses, namely UV-light and heat are analyzed by treating tobacco or Arabidopsis leaf discs. For UV-B resistance, leaf discs are irradiated with 20 W UV-B lamps (32 kJ/m²) supplemented with white fluorescent light (5 mMol/m²/sec). Light intensity is measured by a radiometer. UV damage is assessed by (i) loss of leaf color (chlorophyll content) and (ii) ion leakage, which is determined by conductivity measurements following washing irradiated leaves with deionized water. Leaf samples are taken every 12 hours for two days. In addition, intact plants are phenotypically evaluated after direct UV-B irradiation.

A DNA laddering assay is performed as follows. Treatment of tobacco cotyledons at 55 C for 12 minutes resulted in internucleosomal cleavage of DNA. Transgenic tobacco containing a PAD or tomato BI-1 transgene is evaluated under the same conditions. DNA laddering is prevented by the two cytoprotective genes but not by control DNA sequences.

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

I claim:

1. A non-naturally occurring plant, comprising an ectopically expressed nucleic acid molecule encoding a plant anti-death (PAD) polypeptide or active fragment thereof, said non-naturally occurring plant characterized by increased resistance to biotic or abiotic stress.

2. The non-naturally occurring plant of claim 1, wherein said PAD polypeptide has substantially the amino acid sequence of tomato PAD-1 (SEQ ID NO:2).

3. The non-naturally occurring plant of claim 2, wherein said PAD polypeptide has the amino acid sequence of tomato PAD-1 (SEQ ID NO:2).

4. The non-naturally occurring plant of claim 1, wherein said PAD polypeptide has the amino acid sequence of an ortholog of tomato PAD-1.

5. The non-naturally occurring plant of claim 1, which is a transgenic plant.

6. The transgenic plant of claim 5, wherein said ectopically expressed nucleic acid molecule encoding a PAD polypeptide is operatively linked to an exogenous regulatory element.

7. The transgenic plant of claim 6, said nucleic acid molecule comprising an exogenous nucleic acid molecule encoding a PAD polypeptide having the amino acid sequence of an ortholog of tomato PAD-1.

8. The transgenic plant of claim 6, wherein said exogenous regulatory element is a constitutive regulatory element.

9. The transgenic plant of claim 6, wherein said exogenous regulatory element is an inducible regulatory element.

10. The transgenic plant of claim 5, which is selected from the group consisting of a rice, corn, wheat, soybean, common fruit and ornamental flower plant.

11. The transgenic plant of claim 5, which is a grass.

12. The transgenic plant of claim 11, which is a turf grass.

13. A tissue derived from a transgenic plant, said plant comprising an ectopically expressible nucleic acid molecule encoding a PAD polypeptide and characterized by increased resistance to biotic or abiotic stress.

14. The tissue of claim 13, which is a seed.

15. The tissue of claim 13, which is a fruit.

16. A method of increasing the resistance of a plant to biotic or abiotic stress, comprising ectopically expressing in said plant a nucleic acid molecule encoding a plant anti-death (PAD) polypeptide or active fragment thereof.

17. The method of claim 16, comprising introducing into said plant a nucleic acid molecule encoding a PAD polypeptide or active fragment thereof, thereby increasing the resistance of said plant to biotic or abiotic stress.

18. An isolated polypeptide, comprising an amino acid sequence encoding a plant anti-death (PAD) polypeptide or an active fragment thereof.

19. The isolated polypeptide of claim 18, wherein said PAD polypeptide has substantially the amino acid sequence of tomato PAD-1 (SEQ ID NO: 2).

20. The isolated polypeptide of claim 19, wherein said PAD polypeptide has the amino acid sequence of tomato PAD-1 (SEQ ID NO: 2).

21. The isolated polypeptide of claim 18, wherein said PAD polypeptide has the amino acid sequence of an ortholog of tomato PAD-1.

22. An isolated nucleic acid molecule, comprising a nucleic acid sequence encoding a tomato Bax inhibitor-1 (BI-1) polypeptide or active fragment thereof, provided that said nucleic acid molecule is not GenBank accession AI771102.

23. The isolated nucleic acid molecule of claim 22, wherein said tomato BI-1 polypeptide has substantially the amino acid sequence of tomato BI-1 (SEQ ID NO: 4).

24. The isolated nucleic acid molecule of claim 23, comprising a nucleic acid sequence encoding the amino acid sequence SEQ ID NO: 4.

25. The isolated nucleic acid molecule of claim 24, comprising the nucleic acid sequence SEQ ID NO: 3.

26. A vector, comprising a nucleic acid molecule encoding a tomato Bax inhibitor-1 (BI-1) polypeptide or active fragment thereof, provided that said nucleic acid molecule is not GenBank accession AI771102.

27. The vector of claim 26, which is a plant expression vector.

28. The vector of claim 26, wherein said tomato BI-1 polypeptide has substantially the amino acid sequence of tomato BI-1 (SEQ ID NO: 4).

29. A non-naturally occurring plant, comprising an ectopically expressed nucleic acid molecule encoding a tomato Bax inhibitor-1 (BI-1) polypeptide or active fragment thereof, said non-naturally occurring plant characterized by increased resistance to biotic or abiotic stress.

30. The non-naturally occurring plant of claim 29, wherein said tomato BI-1 polypeptide has substantially the amino acid sequence of tomato BI-1 (SEQ ID NO: 4).

31. The non-naturally occurring plant of claim 30, wherein said tomato BI-1 polypeptide has the amino acid sequence of tomato BI-1 (SEQ ID NO: 4).

32. The non-naturally occurring plant of claim 29, which is a transgenic plant.

33. The transgenic plant of claim 32, wherein said ectopically expressed nucleic acid molecule encoding a tomato BI-1 polypeptide is operatively linked to an exogenous regulatory element.

34. The transgenic plant of claim 33, wherein said exogenous regulatory element is a constitutive regulatory element.

35. The transgenic plant of claim 33, wherein said exogenous regulatory element is an inducible regulatory element.

36. The transgenic plant of claim 32, which is selected from the group consisting of a rice, corn, wheat, soybean, common fruit and ornamental flower plant.

37. The transgenic plant of claim 32, which is a grass.

38. The transgenic plant of claim 37, which is a turf grass.

39. A tissue derived from a transgenic plant, said plant comprising an ectopically expressible nucleic acid molecule encoding a tomato BI-1 polypeptide and characterized by increased resistance to biotic or abiotic stress.

40. The tissue of claim 39, which is a seed.

41. The tissue of claim 39, which is a fruit.

42. A method of increasing the resistance of a plant to biotic or abiotic stress, comprising ectopically expressing in said plant a nucleic acid molecule encoding a tomato Bax inhibitor-1 (BI-1) polypeptide or active fragment thereof.

43. The method of claim 42, comprising introducing into said plant a nucleic acid molecule encoding a tomato BI-1 polypeptide or active fragment thereof, thereby increasing the resistance of said plant to biotic or abiotic stress.

44. An isolated polypeptide, comprising an amino acid sequence encoding tomato BI-1 or an active fragment thereof.

45. The isolated polypeptide of claim 44, wherein said tomato BI-1 has substantially the amino acid sequence of tomato BI-1 (SEQ ID NO: 4).

46. The isolated polypeptide of claim 45, wherein said tomato BI-1 has the amino acid sequence of tomato BI-1 (SEQ ID NO: 4)


SEQUENCE LISTING (7 sequences)

SEQ ID NO: 1 - DNA, 617 bp, Lycopersicon esculentum
  CDS (103)...(594)
cgttctctgatgcgtagctgaagaaatatcaccggaaagttcacaggaaataagtggcggcggtgcgtggctcttgttgaataccaagcgagaggagataagatgccggaacatcctgctgcagactcatcagccaccgacaacaccgtcaccgtcaagcgttatgcccctcccaatcagcggaatcgttcactcggcaggcgaaaatctggagatcgacttgaaagagctagcagctatgctagtgatggagagaagaaccaaatgagagcagctaagtctgtatctgatgctggagtcaatcgagtaaatgattatcctccaacaaagttaataccgctacaaggatgttgtacaagcgaagcttttcagctactaaatgaccgctgggcagctgctctgaatgctcataataatttatcagaagattctcgtgaaaggcctgtaatgtacacaaaaagatcaccttgggggcatccttttcttccacatcaattgatgtcacaagcaggagctgaatcttctactggccagaaggattttctaagcaaacttcagatggctatgctcaatacacatgtcaatttcgatgcctaaatgctatccatcaagtggtc

SEQ ID NO: 2 - protein, 164 aa, Lycopersicon esculentum
MPEHPAADSSATDNTVTVKRYAPPNQRNRSLGRRKSGDRLERASSYASDGEKNQMRAAKSVSDAGVNRVNDYPPTKLIPLQGCCTSEAFQLLNDRWAAALNAHNNLSEDSRERPVMYTKRSPWGHPFLPHQLMSQAGAESSTGQKDFLSKLQMAMLNTHVNFDA

SEQ ID NO: 3 - DNA, 1034 bp, Lycopersicon esculentum
  CDS (87)...(830)
  misc_feature (1)...(1034): n = A,T,C or G
gagcaaacataacattgtctacgttcagataaatatcctttgctcatttcagttccaaaaactcgaagaagaagaagaagagaacaatggaaggtttcacatcgttcttcgactcgcaatctgcctctcgcaaccgctggagttatgattctctcaaaaacttccgccagatctcacctctcgttcaaactcatctcaagcaggtgtaccttacgctatgctgtgctttagtggcatcggctgctggggcttaccttcacattctatggaatatcggtggcctcctcacaacaatggcttgcatgggaagcatggtgtggcttctctcagctcctccttatcaagagcaaaaaagggtggctcttctgatggcagctgcactttttgaaggcgcctctattggtcctctgattgagctgggcattaacttcgatccaagcattgtgtttggcgcttttgtaggttgtgctgtggtttttggttgcttctcagctgctgccatgttggcaaggcgcagggagtacttgtacctcgggggccttctttcatctggcgtctcccttctcttctggttgcactttgcatcctccatttttggtggttccatggctgttttcaagtttgagttgtattttggactcttggtgtttgtgggctacatcgtctttgacacccaagaaattattgagaaggctcacttgggtgatatggattacgttaagcatgcattgacccttttcacagattttggcgctgtttttgtgcggattctgatcatcatgttaaagaatgcatctgagaaggaagagaagaagaagaagaggagaaactagatttgcttctcaacttgtggtttccanaactccttgtgttcacctgaaacaagcatgttaatagtttgatacttgcttcactttagcataggctgtgatgtaatgtcgtgtgacatgccattatggctgtgtgattgagcatctagcctttttatcttctaaagcttttttcttaacattgataaggaaagttccttgtga

SEQ ID NO: 4 - protein, 248 aa, Lycopersicon esculentum
MEGFTSFFDSQSASRNRWSYDSLKNFRQISPLVQTHLKQVYLTLCCALVASAAGAYLHILWNIGGLLTTMACMGSMVWLLSAPPYQEQKRVALLMAAALFEGASIGPLIELGINFDPSIVFGAFVGCAVVFGCFSAAAMLARRREYLYLGGLLSSGVSLLFWLHFASSIFGGSMAVFKFELYFGLLVFVGYIVFDTQEIIEKAHLGDMDYVKHALTLFTDFGAVFVRILIIMLKNASEKEEKKKKRRN

SEQ ID NO: 5 - DNA, 28 bp, Artificial Sequence
  primer
ggaattcatggaaggtttcacatcgttc

SEQ ID NO: 6 - DNA, 43 bp, Artificial Sequence
  primer
ccgctcgagctagggtcgactgtttctcctcttcttcttcttc

SEQ ID NO: 7 - DNA, 28 bp, Artificial Sequence
  primer
ggaattcatgccggaacatcctgctgca